(19) United States
(12) Patent Application Publication
(10) Pub. No.: US 2007/0149979 A1
(43) Pub. Date: Jun. 28, 2007

(54) Targeting device

(76) Inventor: Gursharan Singh Chana, Sutton Coldfield (GB)

Correspondence Address: ICE MILLER LLP, ONE AMERICAN SQUARE, SUITE 3100, INDIANAPOLIS, IN, 46282-0200, US

(21) Appl. No.: 10/596,402

(22) PCT Filed: Dec. 9, 2004
(86) PCT No.: PCT/GB04/05168

§ 371 Date: Jan. 4, 2007

(30) Foreign Application Priority Data
Dec. 12, 2003 (GB) 0328788.5
Feb. 4, 2004 (GB) 0402471.7
Sep. 9, 2004 (GB) 0419983.2

(51) Int. Cl. A61B 17/60

(52) U.S. Cl. 606/96

(57) ABSTRACT

The present invention provides a targeting device (1) for use in open, or minimally invasive, hip surgery to allow the position of the centreline of the femoral neck to be located. Also disclosed is its use in location of the centreline of the femoral neck. The targeting device comprises at least a first component (4) having a portion suitable for location on an outer surface of the femoral neck and a second component (7) having a portion suitable for marking the centreline of the femoral neck, wherein the first (4) and second (7) components are spaced apart from and parallel to one another and means is provided to alter the distance between the first (4) and second (7) components and means is provided to maintain the first (4) and second (7) components in a predetermined position relative to each other.

The present invention relates to an improved targeting device, particularly for use in hip surgery and more particularly for use in hip replacement surgery or hip resurfacing surgery, and its use in surgery, particularly minimally invasive surgery.

When performing hip replacement surgery it is essential to be able to accurately determine where the centre of the base of the femoral neck and the centreline through the femoral neck lie to allow correct reaming fitting of the replacement head.

The position of the femoral head does not always assist in determining the position of the centre of the femoral neck and the centreline through the femoral neck as the head may not be centrally positioned in relation to the femoral neck due to deformity of the femoral head.

When performing hip resurfacing surgery it is essential to be able to accurately determine where the centreline through the femoral neck and femoral head lies to allow correct reaming of the femoral head and correct fitting of the prosthesis onto the femoral head.

The position of the femoral head does not always assist in determining the position of the centreline through the femoral neck and femoral head as the head may not be centrally positioned in relation to the femoral neck due to deformity of the femoral head.

Accordingly there is the need for a targeting device which is suitable to allow the position of the centre of the femoral neck and/or the centreline through the femoral neck and femoral head (hereinafter reference to “the centreline of the femoral neck” is used to cover both of these references) to be determined, which device can be used in minimally invasive surgical methods and in conventional open surgical methods.

The present invention provides in a first aspect a targeting device for use in open, or minimally invasive, hip surgery to allow the position of the centreline of the femoral neck to be located, which device comprises at least a first component having a portion suitable for location on an outer surface of the femoral neck and a second component having a portion suitable for marking the centreline of the femoral neck, wherein the first and second components are spaced apart from and parallel to one another and means is provided to alter the distance between the first and second components and means is provided to maintain the first and second components in a predetermined position relative to each other.

The present invention provides in a first embodiment a targeting device for use in open, or minimally invasive, hip surgery to allow the position of the centreline of the femoral neck to be located, which device comprises at least a first component having a portion suitable for receipt of a first guide wire and/or a second component having a portion suitable for receipt of a second guide wire, wherein the first and second components are spaced apart from and parallel to one another and means is provided to alter the distance between the first and second components and means is provided to maintain the first and second components in a predetermined position relative to each other.

The first and second components may be identical in configuration.

Preferably each of the first and second components includes an elongate three dimensional shape, such as a cylinder, for location on an outer surface of the femoral neck or for marking the centreline of the femoral neck. Each three dimensional shape of the first and second components is preferably 2 to 8 cm in length, more preferably 3 to 6 cm in length and most preferably 4 to 5 cm in length.

A central bore is preferably provided along the centre of each of the elongate three dimensional shapes of the first and second components to receive a guide wire. The bore may have a diameter of 1.5 to 5 mm, preferably 3 to 4 mm, most preferably 3.2 to 3.5 mm.

Each of the elongate three dimensional shapes of the first and second components is most preferably cylindrical in shape with a central bore extending along its length, i.e. each of the three dimensional elongate shapes of the first and second components is tubular.

The means provided to space the first and second components from each other and maintain the first and second components parallel to each other may be any suitable means.

Preferably the first component is provided with one or more runners, the or each runner is elongate and extends such that its longitudinal axis is perpendicular to that of the first component. The or each runner is preferably secured, for example by welding, to the first component. Where there are two or more runners the runners are parallel to each other. The or each runner may be up to 6 cm in length, preferably up to 5 cm and most preferably up to 3 or 2 cm.

The second component is preferably provided with means to movably, preferably slidably, engage the or each runner. The second component is positioned on the or each runner such that the longitudinal axis of the second component lies perpendicular to that of the or each runner and parallel to the longitudinal axis of the first component. In use the second component is movable along the or each runner to alter the distance between the first and second component.

Alternatively the second component is provided with one or more runners, the or each runner is elongate and extends such that its longitudinal axis is perpendicular to that of the second component. The or each runner is preferably secured, for example by welding, to the second component Where there are two or more runners the runners are parallel to each other. The or each runner may be up to 6 cm in length, preferably up to 5 cm and most preferably up to 3 or 2 cm.

The first component is preferably provided with means to movably, preferably slidably, engage the or each runner. The first component is positioned on the or each runner such that the longitudinal axis of the first component lies perpendicular to that of the or each runner and parallel to the longitudinal axis of the first component. In use the second component is movable in relation to the first component by the or each runner moving in relation to the first component. This arrangement ensures that the device is compact and when the first and second components are close together the or each runner extend out of the incision to avoid tissue damage.

One or more springs may be provided between the first and second components to assist movement of the second component away from the first component, preferably the or each spring is provided around the or each runner.

In a most preferred embodiment each of the first and second components is provided with a mounting portion and an elongate three dimensional shaped portion. The mounting portion is suitably sized and shaped to receive or co-operate with the elongate three dimensional shaped portion, the or each runner and the means provided to space the first and second components from each other and maintain the first and second components parallel to each other. The elongate three dimensional shaped portions are preferably releasably secured to the mounting portions, most preferably each of the elongate three dimensional shaped portions is tubular and has an external screw threaded portion. The mounting portions preferably each have one or more apertures with corresponding internal screw threaded portions to receive and hold a tubular portion. This allows the tubular portions to be removed for cleaning or to be disposed of and replaced.

The targeting device may also be provided with a repositioning device. The repositioning device preferably comprises a plate to be secured to the targeting device and having one, two or more tubular components, sized to receive a guide wire, extending therefrom. The plate is most preferably received by the apertures in the first and second components when the first and second components are positioned as close together as possible. This fixing is quick and stable.

The means to alter the distance between the first and second components may be any suitable means. The distance between the first and second components could be up to 3 cm, preferably up to 2.5 cm.

The means to maintain the position of the second component relative to the first component may be any suitable means and is preferably the same means as the means to alter the distance between the first and second components.

Preferably there is provided a screw threaded bar received by corresponding screw threaded portions on each of the first and second components. The portions of the first and second components receiving the bar are preferably positioned on a common axis extending perpendicular to and between the first and second components. The portions of first and second components receiving the bar are preferably protrusions having corresponding screw threaded apertures therethrough.

The screw threaded bar is preferably provided with a suitable means to cause its rotation, for example a knob.

In a most preferred embodiment the longitudinal axes of the or each runner and the screw threaded bar all lie parallel to the longitudinal axis of the elongate support and the longitudinal axes of the first and second components each lie perpendicular to these axes and are spaced apart from and parallel to each other.

The first component is preferably mounted on an elongate support. The elongate support preferably has a handle portion distal from the first component. The first component is preferably mounted on the elongate support perpendicular to the longitudinal axis of the elongate support.

Alternatively the first component is mounted on an elongate support and the second component is mounted on a bar telescopically received within the elongate support. The bar is preferably slidably moved into and out of the elongate support by means of a screw thread system.

Preferably a rotatable shaft is provided within the elongate support having a screw thread corresponding to a screw thread provided on the telescopically received bar. The screw thread on the bar may be external and the screw thread on the shaft may be internal or vice versa. The rotatable shaft is preferably provided with a knob to assist in its rotation. Rotation of the rotatable shaft causes movement of the telescopically received bar into and out of the elongate support. When rotation of the rotatable shaft is stopped the first and second components are held in the desired position relative to each other.

The screw thread system is preferably provided with a suitable means to cause the bar to move into and out of the elongate support by rotation, for example a knob.

In a most preferred embodiment the longitudinal axes of the or each runner the elongate support and the telescopically received bar all lie parallel to the longitudinal axis of the elongate support and the longitudinal axes of the first and second components each lie perpendicular to these axes and are spaced apart from and parallel to each other.

The device may comprise a first component and a second component only. Alternatively the device may further include at least a third component. The third component is preferably an “L” shaped bar having a first elongate portion and a second elongate portion, the second elongate portion extending generally perpendicularly to the first elongate portion. The length of the first elongate portion is greater than that of the second elongate portion.

The “L” shaped bar may be any suitable cross section but is preferably of circular, square or rectangular cross section, the “L” shaped bar is preferably of a cross section that prevents its rotation around its longitudinal axis to ensure that a desired alignment is maintained. The “L” shaped bar may be solid or hollow.

The length of the first elongate portion may be from 10 to 30 cm in length, preferably from 15 to 25 cm and most preferably from 15 to 20 cm.

The “L” shaped bar is positioned parallel to and spaced apart from the first component. The “L” shaped bar is preferably mounted on the elongate support on the side of the first component opposite to that of the second component.

The provision of the “L” shaped bar is particularly advantageous in hip resurfacing surgery where it ensures that the first component is in line with the outer surface of the femoral neck. Without the provision of the “L” shaped bar this cannot be done accurately as the femoral head is in the way and it is not therefore possible to see whether the first component is actually positioned in line with the outer surface of the femoral neck. In hip replacement surgery the provision of the “L” shaped bar provides additional assistance in locating and steadying the targeting device.

The “L” shaped bar is mounted on the elongate support such that the first elongate portion is parallel to the first component and the “L” shaped bar and the “L” shaped bar is movable along the longitudinal axis of the first elongate portion.

The “L” shaped bar being movable along the longitudinal axis of the first portion allows for its use with different sized hip joints by allowing for the device to be used around different sized femoral heads.

The “L” shaped bar is mounted on the elongate support such that the free end of the second elongate portion extends as far as the longitudinal axis through the first component. The free end of the second elongate portion may be provided with a plate perpendicular thereto which lies along the longitudinal axis through the first component. Accordingly the length of the second elongate portion, including any plate, is the same as the distance between the first component and the first elongate portion of the

“L” shaped bar, this distance is preferably 2 to 5 cm, most preferably 2 to 3 cm.

The present invention provides in a second embodiment a targeting device for use in open, or minimally invasive, hip surgery to allow the position of the centreline of the femoral neck to be located, wherein the first component is an “L” shaped bar according to the first embodiment and the second component is a second component according to the first embodiment.

Preferably the second component is movable in relation to the “L” shaped bar such that the second component is spaced apart from the free end of the second elongate portion of the “L” shaped bar by a distance that is half of the diameter of the femoral neck to allow the centreline through the femoral neck to be determined.

The targeting device may be manufactured from any suitable material, for example metal such as stainless steel or surgical steel or a plastics material.

In a second aspect the targeting device of the present invention may be used in a method of locating the centre of the osteotomised base of the femoral neck at the head-neck junction, which method comprises:
measuring the diameter of the femoral neck;
dividing the diameter of the femoral neck by two to give value X and setting the distance between the first and second components of the targeting device as X;
determining the mid line through the femoral neck in the AP plane and running a guide wire along this line through the first component of the targeting device of the present invention.

Preferably a second guide wire is passed through the second component to mark the centre of the osteotomised femoral neck. The targeting device may then be removed leaving at least the second guide wire in place.

Preferably the diameter of the femoral neck is measured using callipers. Alternatively the diameter may be measured using a suitable calibrated gauge.

The “L” shaped bar may be provided and positioned such that the free end of the second elongate portion of the “L” shaped bar rests on the femoral neck on the midline in the AP plane. This assists in location and use of the targeting device.

In a third aspect the targeting device of the present invention may be used in a method of locating the centreline through the femoral neck and femoral head, which method comprises:
measuring the diameter of the femoral neck;
dividing the diameter of the femoral neck by two to give value X and setting the distance between the first and second components of the targeting device as X;
determining the mid line through the femoral neck in the AP plane;
positioning the free end of the second elongate portion of the “L” shaped bar on the femoral neck on the midline in the AP plane;
determining the centreline through the femoral neck and femoral head by position of the second component.

A guide wire may be passed through the first component once its position has been determined using the “L” shaped bar.

Preferably a second guide wire is passed through the second component to mark the central longitudinal axis of the femoral neck. The targeting device may then be removed leaving at least the second guide wire in place.

Preferably the diameter of the femoral neck is measured using callipers. Alternatively the diameter may be measured using a suitable calibrated gauge.

The present invention further provides a gauge for measuring the diameter of a femoral neck, which gauge comprises a first component and a second component wherein at least one of the first and second components is movable relative to the other said component and means is provided to determine the distance between the first and second components.

Preferably the first component is fixed in position and the second component is movable in relation to the first component or vice versa.

Preferably the device is provided with a handle portion to which the first component is fixedly mounted, preferably by means of a generally semi-circular joining member.

The second component is preferably slidably mounted on or within the handle portion and is movable towards and away from the second component.

The distance between the first and second component may be determined by any suitable means, for example by the provision of a scale to show the distance between the first and second component depending on the relative positions of the first and second components.

A specific embodiment of the present invention will now be described, by means of example only, with reference to the drawings, in which:

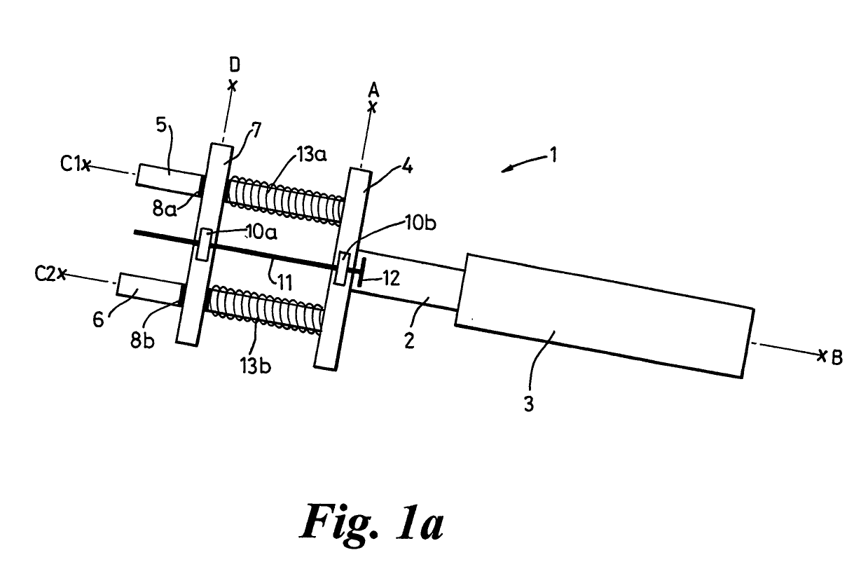

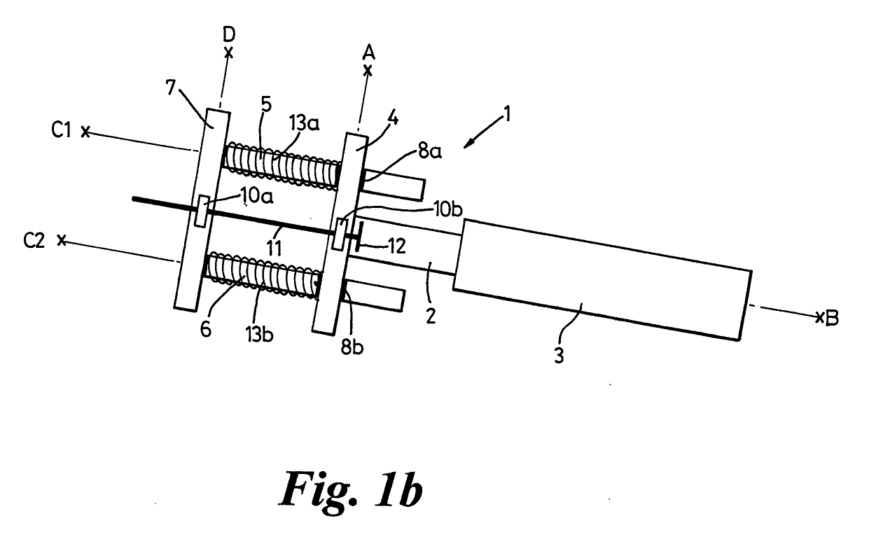

FIGS. 1a, 1b and 1c show, in plan view, schematic drawings of a first, second and third targeting device according to the present invention;

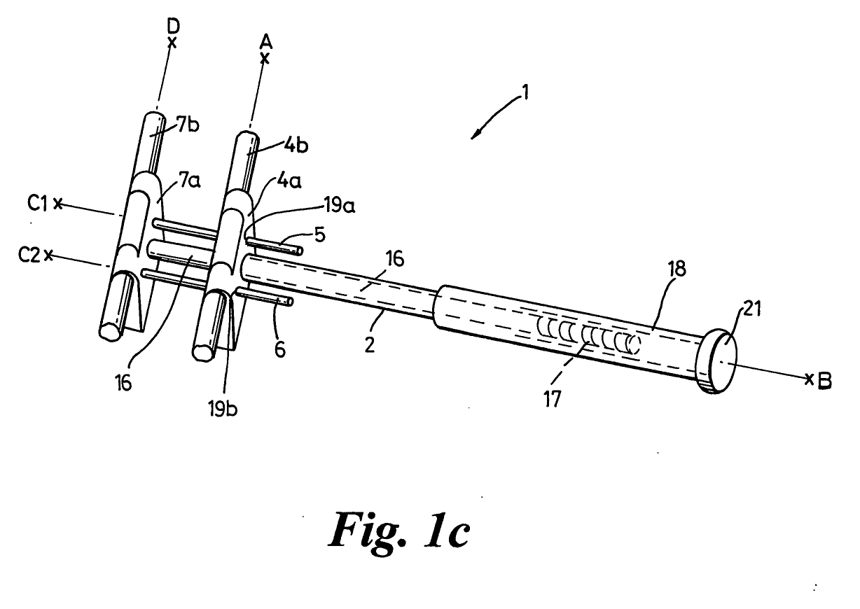

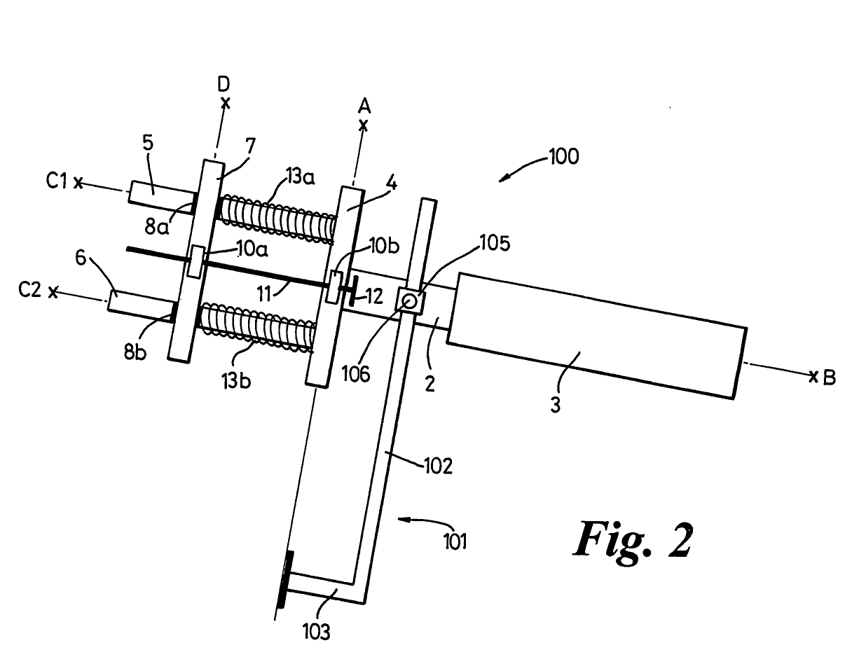

FIG. 2 shows, in plan view, a schematic drawing of a further targeting device according to the present invention; and

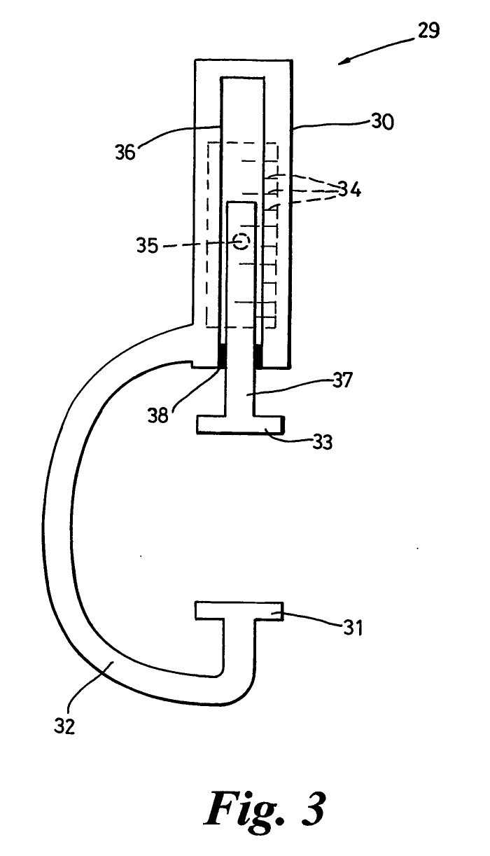

FIG. 3 shows a cross section through a gauge for measuring the diameter of a femoral neck.

FIGS. 1a and 1b shows a targeting device 1 suitable for use in open, or minimally invasive, hip replacement surgery. The device 1 comprises an elongate support 2 having a handle portion 3 at one end having a central longitudinal axis B. Secured to the end of the elongate support distal from the handle portion 3, by welding, is a first component 4 suitable for receiving a guide wire.

In a first embodiment, shown in FIG. 1a, the first component 4 comprises a tube having a central longitudinal axis A lying perpendicular to the longitudinal axis B of the elongate support 2.

Two elongate runners 5, 6 are provided extending tangentially to the first component 4. The runners 5, 6 are parallel to and spaced apart from each other and their central longitudinal axes C1, C2 run parallel to axis B of the elongate support 2. The runners 5, 6 are secured to the first component 4 by welding.

A second component 7 is slidably mounted on the runners 5, 6. The second component 7 comprises a tube having a central longitudinal axis D parallel to axis A of the first component 4.

The second component 7 is provided with two suitably sized shoulders 8a, 8b having apertures 9a, 9b (not shown) therein to receive the runners 5, 6. The shoulders 8a, 8b are positioned such that the runners form an identical tangent to the second component 7 as they do to the first component 4.

Each of the first 4 and second 7 components is provided with an integral protrusion 10a, 10b positioned diametrically opposite to the runners 5, 6 and having a screw threaded aperture therethrough. A correspondingly screw threaded bar 11 is provided extending through the apertures in the protrusions 10a, 10b and rotation of this bar 11 causes movement of the second component 7 relative to the first component 4 along the runners 5, 6. The bar 11 is provided with a knob 12 to assist in its manual rotation.

In a second embodiment, shown in FIG. 1b, the first component 4 comprises a tube having a central longitudinal axis A lying perpendicular to the longitudinal axis B of the elongate support 2. A second component 7 is provided and comprises a tube having a central longitudinal axis D parallel to axis A of the first component 4.

Two elongate runners 5, 6 are provided extending tangentially to the first component 4. The runners 5, 6 are parallel to and spaced apart from each other and their central longitudinal axes C1, C2 run parallel to axis B of the elongate support 2. The runners 5, 6 are secured to the second component 7 by welding.

The first component 4 slidably receives the runners 5, 6 by provision of with two suitably sized shoulders 8a, 8b having apertures 9a, 9b (not shown) therein to receive the runners 5, 6. The shoulders 8a, 8b are positioned such that the runners form an identical tangent to the first component 4 as they do to the second component 7.

Each of the first 4 and second 7 components is provided with an integral protrusion 10a, 10b positioned diametrically opposite to the runners 5, 6 and having a screw threaded aperture therethrough. A correspondingly screw threaded bar 11 is provided extending through the apertures in the protrusions 10a, 10b and rotation of this bar 11 causes movement of the second component 7 relative to the first component 4 by the runners 5, 6 sliding through the apertures 9a and 9b in shoulders 8a, 8b. The bar 11 is provided with a knob 12 to assist in its manual rotation.

In both embodiments springs 13a, 13b are provided around the runners 5, 6 between the first 4 and second 7 components to assist movement of the second component 7 away from the first component 4.

In a third embodiment, shown in FIG. 1c, the first component 4 includes a mounting portion 4a and a tube 4b having a central longitudinal axis A lying perpendicular to the longitudinal axis B of the elongate support 2. A second component 7 is provided and includes a mounting portion 7a and a tube 7b having a central longitudinal axis D parallel to axis A of the first component 4. The tubes 4b and 7b have external screw threaded portions (not shown) and are releasably secured to the mounting portions 4a and 7a by the provision of apertures in the mounting portions with internal screw threaded portions (not shown) corresponding to external screw threads of the tubes 4b, 7b. The tubes 4b, 7b can then be removed for cleaning or can be disposed of and replaced.

The mounting portion 4a of the first component 4 is secured to the elongate support 2 by welding. The mounting portion 7a of the second component 7 is secured to a bar 16 by welding. The bar 16 is telescopically received by the elongate support 2 which has a bore along its length.

Two elongate runners 5, 6 are provided extending from the mounting portion 7a of the second component 7. The runners 5, 6 are parallel to and spaced apart from each other and their central longitudinal axes C1, C2 run parallel to axis B of the elongate support 2. The runners 5, 6 are secured to the second component 7 by welding.

The mounting portion 4a of the first component 4 slidably receives the runners 5, 6 by provision of two suitably sized apertures 19a, 19b therein to receive the runners 5, 6.

The second component 7 is moved relative to the first component 4 by the bar 16 telescopically moving into and out of the bore in the elongate support 2. During this movement the runners 5, 6 hold the position of the second component and prevent the bar 16 from rotating. To cause movement of the bar 16 along the bore of the elongate support 2 the bar 16 is provided with an external screw thread 17 around its end positioned farthest from the second component. The elongate support 2 is provided with a rotatable shaft 18 positioned within the elongate support and having an internal screw thread (not shown) corresponding to the external screw thread of the bar 16. The rotatable shaft is provided with a knob 21 at the end thereof, and positioned on the outside of the elongate support 2, to allow rotation of the shaft 18. The rotation of the shaft 18 causes movement of the bar 16 by means of the action of the corresponding screw threads and the prevention of rotation of the bar 16 by means of the runners 5, 6.

In use the targeting devices of the present invention assist in locating the central longitudinal axis of the femoral neck. In minimally invasive hip replacement surgery as described in International Patent Publication No WO 03/065906 of the same inventor, or in open hip replacement surgery, the diameter of the femoral neck is measured and the radius is calculated by dividing the diameter by two.

The distance of the centre of the second component 7 from the centre of the first component 4 is adjusted by rotation of the screw threaded bar 11 until the distance is the same as the measured diameter divided by two.

The centre line of the femoral neck in the AP plane is determined and a first guide wire is inserted in line with this centre line. The first guide wire is passed through the first component 4 of the targeting device 1. The second component 7 of the targeting device is then used to locate the centre in the AP and lateral plane by rotation around the first guide wire. The centre is then marked with a second guide wire and the targeting device can be removed leaving the second guide wire in place.

The femoral neck can then be reamed accurately and centrally and the resultant prosthesis will be accurately and centrally positioned.

FIG. 2 shows a further targeting device 100 suitable for use in open, or minimally invasive, hip surgery, especially minimally invasive hip resurfacing surgery. In FIG. 2 the targeting device 100 is similar to targeting device 1 of the embodiment of FIG. 1a and like numbers relate to like parts.

Targeting device 100 is additionally provided with an “L” shaped bar 101 comprising a first elongate portion 102 and a second elongate portion 103 where the first elongate portion is of greater length than the second elongate portion.

The “L” shaped bar 101 is mounted on the elongate support 2 such that the first elongate portion 102 is parallel to the first component 4. The second elongate portion 103 is perpendicular to the first elongate portion 102 and the free end of the second portion is provided with a plate 104 that lies parallel with the first component 4.

The “L” shaped bar 101 is sized and mounted on the elongate support 2 such that the plate 104 lies along the longitudinal axis through the first component 4.

The “L” shaped bar 101 is movable along the longitudinal axis through the first elongate portion 102. The “L” shaped bar 101 is mounted on the elongate support 2 by means of a channel (not shown) provided through a block 105 secured by welding to the elongate support 2 in which the first elongate portion 102 is received. A bolt 106 is provided extending into the channel through the block 105 to contact the first elongate portion and lock the “L” shaped bar in a desired position.

The “L” shaped bar can also be used with the targeting device of FIGS. 1b and 1c in the same manner as described in relation to FIG. 1a

In use the targeting device of the present invention assists in locating the central longitudinal axis of the femoral neck. In minimally invasive hip resurfacing surgery as described in International Patent Publication No WO 03/065906 of the same inventor the femoral head can be delivered into the wound at 90° of internal rotation, 45° of flexion and as much adduction as possible as described. To locate the centreline through the femoral neck and femoral head, in the AP and lateral plane, in open, or minimally invasive, hip resurfacing surgery the diameter of the femoral neck is measured and the radius is calculated by dividing the diameter by two.

The distance of the centre of the second component 7 from the centre of the first component 4 is adjusted by rotation of the screw threaded bar 11 until the distance is the same as the measured diameter divided by two.

The centre line of the femoral neck in the AP plane is determined and the plate 104 of the “L” shaped bar 101 is positioned on the centreline in the AP plane. A first guide wire may be passed through the first component 4 of the targeting device 1 and this is certain to be in line with the centreline in the AP plane owing to the use of the “L” shaped bar. The second component 7 of the targeting device is used to locate the centre in the AP and lateral plane by rotation around the first guide wire and/or the “L” shaped bar. The centre is then marked with a guide wire passed through the second component and the targeting device can be removed leaving the second guide wire in place.

The femoral head can then be reamed accurately and centrally and the resultant prosthesis will be accurately and centrally positioned.

FIG. 3 shows a gauge for use in measuring the diameter of a femoral head or neck. The gauge 29 comprises an elongate handle 30 having a first component 31 fixedly secured thereto such that the centre of the first component lies on the longitudinal axis through the handle 30. The first component is fixed to the handle by means of a semi-circular joining member 32 extending between the handle 30 and the first component 31. The semi-circular joining member 32 allows the device to be used around a generally cylindrical bone to determine the diameter thereof.

A second component 33 is provided slidably attached within a channel 36 in the handle 30 by a rod 37 and seal 38 arrangement. The second component also lies on the longitudinal axis through the handle and is positioned between the handle 30 and the first component 31. A measuring scale 34 is provided (shown in dotted lines), calibrated to show the distance between the first and second components as the second component moves in relation to the first component. A marker 35 (shown in dotted lines) secured to the second component 33 moves along the scale as the second component 33 moves in relation to the first component 31 showing the distance between the two components.

The device is sized such that the maximum distance between the two components is 4 cm.

1. A targeting device for use in open, or minimally invasive, hip surgery to allow the position of the centreline of the femoral neck to be located, which device comprises at least a first component having a portion suitable for location on an outer surface of the femoral neck and a second component having a portion suitable for marking the centreline of the femoral neck, wherein the first and second components are spaced apart from and parallel to one another and means is provided to alter the distance between the first and second components and means is provided to maintain the first and second components in a predetermined position relative to each other and wherein one or both of the first component and the second component has a portion suitable for receipt of a guide wire.

2. A targeting device according to claim 1 wherein the first component has a portion suitable for receipt of a first guide wire and the second component has a portion suitable for receipt of a second guide wire.

3. A targeting device according to claim 1 wherein the first component has a portion suitable for receipt of a first guide wire or the second component has a portion suitable for receipt of a second guide wire.

4. A targeting device according to claim 1 wherein the first and second components are identical in configuration.

5. A targeting device according to claim 1 wherein each of the first and second components includes an elongate three dimensional shape for location on an outer surface of the femoral neck or for marking the centreline of the femoral neck.

6. A targeting device according to claim 5 wherein the elongate three dimensional shape is a cylinder.

7. A targeting device according to claim 5 wherein each three dimensional shape of the first and second components is 2 to 8 cm in length.

8. A targeting device according to claim 5 wherein a central bore is provided along the centre of each of the elongate three dimensional shapes of the first and second components to receive a guide wire.

9. A targeting device according to claim 8 wherein the bore has a diameter of 1.5 to 5 mm.

10. A targeting device according to claim 1 wherein the first component is provided with one or more runners, the or each runner is elongate and extends such that its longitudinal axis is perpendicular to that of the first component.

11. A targeting device according to claim 10 wherein the second component is provided with means to movably engage the or each runner.

12. A targeting device according to claim 1 wherein the second component is provided with one or more runners, the or each runner is elongate and extends such that its longitudinal axis is perpendicular to that of the second component.

13. A targeting device according to claim 12 wherein the first component is provided with means to movably engage the or each runner.

14. A targeting device according to claim 10 wherein where there are two or more runners and the runners are parallel to each other.

15. A targeting device according to claim 10 wherein the or each runner is up to 6 cm in length.

16. A targeting device according to claim 1 wherein one or more springs is provided between the first and second components to assist movement of the second component away from the first component.

17. A targeting device according to claim 1 wherein each of the first and second components is provided with a mounting portion and an elongate three dimensional shaped portion and the mounting portion is suitably sized and shaped to receive or co-operate with the elongate three dimensional shaped portion, the or each runner and the means provided to space the first and second components from each other and maintain the first and second components parallel to each other.

18. A targeting device according to claim 17 wherein the elongate three dimensional shaped portions are releasably secured to the mounting portions.

19. A targeting device according to claim 18 wherein the elongate three dimensional shaped portion have an external screw threaded portion and the mounting portions each have one or more apertures with corresponding internal screw threaded portions to receive and hold the elongate three dimensional portion.

20. A targeting device according to claim 1 wherein the targeting device is provided with a repositioning device comprising a plate to be secured to the targeting device and having one, two or more tubular components, sized to receive a guide wire, extending therefrom.

21. A targeting device according to claim 1 wherein the means provided to alter the distance between the first and second components comprises provision of a screw threaded bar received by corresponding screw threaded portions on each of the first and second components.

22. A targeting device according to claim 21 wherein the screw threaded bar is provided with a suitable means to cause its rotation.

23. A targeting device according to claim 21 wherein the first component is provided with one or more runners, the or each runner is elongate and extends such that its longitudinal axis is perpendicular to that of the first component and wherein the longitudinal axes of the or each runner and the screw threaded bar all lie parallel to the longitudinal axis of the elongate support and the longitudinal axes of the first and second components each lie perpendicular to these axes and are spaced apart from and parallel to each other.

24. A targeting device according to claim 1 wherein the first component is mounted on an elongate support.

25. A targeting device according to claim 24 wherein the elongate support has a handle portion distal from the first component.

26. A targeting device according to claim 24 wherein the first component is mounted on the elongate support perpendicular to the longitudinal axis of the elongate support.

27. A targeting device according to claim 24 wherein the first component is mounted on an elongate support and the second component is mounted on a bar telescopically received within the elongate support.

28. A targeting device according to claim 24 wherein the bar is slidably moved into and out of the elongate support by means of a screw thread system.

29. A targeting device according to claim 28 wherein a rotatable shaft is provided within the elongate support having a screw thread corresponding to a screw thread provided on the telescopically received bar.

30. A targeting device according to claim 29 wherein the first component is provided with one or more runners, the or each runner is elongate and extends such that its longitudinal axis is perpendicular to that of the first component and wherein the longitudinal axes of the or each runner, the elongate support and the telescopically received bar all lie parallel to the longitudinal axis of the elongate support and the longitudinal axes of the first and second components each lie perpendicular to these axes and are spaced apart from and parallel to each other.

31. A targeting device according to claim 1 wherein the device further includes at least a third component.

32. A targeting device according to claim 31 wherein the third component is an “L” shaped bar having a first elongate portion and a second elongate portion, the second elongate portion extending generally perpendicularly to the first elongate portion.

33. A targeting device according to claim 32 wherein the length of the first elongate portion is greater than that of the second elongate portion.

34. A targeting device according to claim 32 wherein the “L” shaped bar is of square or rectangular cross section.

35. A targeting device according to claim 32 wherein the length of the first elongate portion is from 10 to 30 cm

36. A targeting device according to claim 32 wherein the “L” shaped bar is positioned parallel to and spaced apart from the first component.

37. A targeting device according to claim 36 wherein the first component is mounted on an elongate support and wherein the “L” shaped bar is mounted on the elongate support on the side of the first component opposite to that of the second component.

38. A targeting device according to claim 37 wherein the “L” shaped bar is mounted on the elongate support such that the first elongate portion is parallel to the first component and the “L” shaped bar is movable along the longitudinal axis of the first elongate portion.

39. A targeting device according to claim 37 wherein the “L” shaped bar is mounted on the elongate support such that the free end of the second elongate portion extends as far as the longitudinal axis through the first component.

40. A targeting device according to claim 39 wherein the free end of the second elongate portion is provided with a plate perpendicular thereto which lies along the longitudinal axis through the first component.

41. A targeting device according to claim 40 wherein the length of the second elongate portion, including any plate, is the same as the distance between the first component and the first elongate portion of the “L” shaped bar and this distance is 2 to 5 cm.

42. A targeting device according to claim 1 wherein the first component is an “L” shaped bar having a first elongate portion and a second elongate portion, the second elongate portion extending generally perpendicular to the first elongate portion and the second component is a second component according to the first embodiment.

43. A targeting device according to claim 42 wherein the second component is movable in relation to the “L” shaped bar such that the second component is spaced apart from the free end of the second elongate portion of the “L” shaped bar by a distance that is half of the diameter of the femoral neck to allow the centreline through the femoral neck to be determined.

44. A method of locating the centre of the osteotomised base of the femoral neck at the head-neck junction, which method comprises:
providing a targeting device for use in open, or minimally invasive, hip surgery to allow the position of the centreline of the femoral neck to be located, which device comprises at least a first component having a portion suitable for location on an outer surface of the femoral neck and a second component having a portion suitable for marking the centreline of the femoral neck, wherein the first and second components are spaced apart from and parallel to one another and means is provided to alter the distance between the first and second components and means is provided to maintain the first and second components in a predetermined position relative to each other and wherein one or both of the first component and the second component has a portion suitable for receipt of a guide wire;
measuring the diameter of the femoral neck;
dividing the diameter of the femoral neck by two to give value X and setting the distance between the first and second components of the targeting device as X;
determining the mid line through the femoral neck in the AP plane and running a guide wire along this line through the first component of the targeting device of the present invention.

45. A method according to claim 44 wherein a second guide wire is passed through the second component to mark the centre of the osteotomised femoral neck.

46. A method according to claim 45 wherein the targeting device is then removed leaving at least the second guide wire in place.

47. A method according to claim 44 wherein an “L” shaped bar is provided and positioned such that the free end of the second elongate portion of the “L” shaped bar rests on the femoral neck on the midline in the AP plane.

48. A method of locating the centreline through the femoral neck and femoral head, which method comprises:
providing a targeting device for use in open, or minimally invasive, hip surgery to allow the position of the centreline of the femoral neck to be located, which device comprises at least a first component having a portion suitable for location on an outer surface of the femoral neck and a second component having a portion suitable for marking the centreline of the femoral neck, wherein the first and second components are spaced apart from and parallel to one another and means is provided to alter the distance between the first and second components and means is provided to maintain the first and second components in a predetermined position relative to each other and wherein one or both of the first component and the second component has a portion suitable for receipt of a guide wire, and wherein the device further includes an “L” shaped bar having a first elongate portion and a second elongate portion, the second elongate portion extending generally perpendicularly to the first elongate portion;
measuring the diameter of the femoral neck;
dividing the diameter of the femoral neck by two to give value X and setting the distance between the first and second components of the targeting device as X;
determining the mid line through the femoral neck in the AP plane;
positioning the free end of the second elongate portion of the “L” shaped bar on the femoral neck on the midline in the AP plane;
determining the centreline through the femoral neck and femoral head by position of the second component.

49. A method according to claim 48 wherein a guide wire is passed through the first component once its position has been determined using the “L” shaped bar.

50. A method according to claim 49 wherein a second guide wire is passed through the second component to mark the central longitudinal axis of the femoral neck.

51. A method according to claim 50 wherein the targeting device is then removed leaving at least the second guide wire in place.

52. A method according to claim 48 wherein the diameter of the femoral neck is measured using callipers or a suitable calibrated gauge.

53.-58. (canceled)
